(19) United States
(12) Patent Application Publication
(10) Pub. No.: US 2026/0209810 A1
(43) Pub. Date: Jul. 23, 2026

(54) METHOD FOR EFFICIENTLY CRYSTALLIZING A-TYPE STARCH

(71) Applicant: CHINA AGRICULTURAL UNIVERSITY, Beijing (CN)

(72) Inventors: Yao HU, Beijing (CN); Fazheng REN, Beijing (CN); Pengjie WANG, Beijing (CN); Ran WANG, Beijing (CN); Siyuan LIU, Beijing (CN); Jiayue GUO, Beijing (CN); Lijuan WANG, Beijing (CN); Yixuan LI, Beijing (CN); Bing FANG, Beijing (CN); Chong CHEN, Beijing (CN); Weibo ZHANG, Beijing (CN)

(21) Appl. No.: 19/566,211

(22) Filed: Mar. 13, 2026

(30) Foreign Application Priority Data
Mar. 26, 2025 (CN) 2025103614056

(51) Int. Cl. C12P 19/14; C08B 31/00; C12P 19/04

(52) U.S. Cl. CPC C12P 19/14; C08B 31/00; C12P 19/04

(57) ABSTRACT

A method for efficiently crystallizing A-type starch includes: gelatinizing and enzymatically debranching starch to obtain an enzymatic hydrolysate of starch; then pumping vapor of a solvent (ethanol, n-butanol, acetone, or the like) into the enzymatic hydrolysate of starch to induce starch crystallization, and centrifuging and drying to obtain A-type crystalline starch. The method of the present disclosure improves the efficiency of A-type starch crystallization, simplifies the preparation process of resistant starch, and shortens preparation time. The A-type crystalline starch prepared by the present disclosure has resistant starch content of more than 68% and a crystallinity of more than 75%.

### TECHNICAL FIELD

The present disclosure relates to a method for efficiently crystallizing A-type starch, belonging to the field of starch crystallization.

### BACKGROUND

Resistant starch refers to starch that resists digestive enzymatic hydrolysis and exhibits the characteristics of dietary fiber. Resistant starch cannot be digested or absorbed in the small intestine of human body, but can be fermented in the large intestine to generate short-chain fatty acids and metabolites beneficial to the human body. Resistant starch is conducive to regulating intestinal health, causing a small fluctuation in blood glucose after being ingested by the human body. The content of resistant starch in most common natural starches is low, and the content of resistant starch in starch extracted from only some special varieties of plants is relatively high, but such starch is typically costly and still requires further processing to meet application requirements in resistant starch content.

Generally, the content of resistant starch in starch is closely related to a crystalline region of starch. Promoting the formation of a crystalline structure in common natural starch through recrystallization is a key technology for industrial processing of resistant starch. Starch crystals are divided into A-type, B-type, V-type, and amorphous crystals, where unit cells of A-type crystals are arranged in the most compact manner and have the strongest anti-digestibility.

Currently, conventional methods for preparing crystalline starch have the deficiencies of cumbersome process, high cost, lack of environmental friendliness, and non-ideal crystallization effect, and the prepared crystalline starch is not A-type, but mostly B-type, V-type, or amorphous. For example,

Chinese patent application No. CN115011652B discloses a method for preparing resistant starch by using vermicelli by-products. According to the method, resistant starch is prepared through recrystallization after enzymatic hydrolysis of starch, recrystallization requires at least 12 hours, B-type crystals of crystalline starch are generated, and toughening treatment is still required after recrystallization to improve the crystallinity of crystalline starch.

Chinese patent application No. CN117099942A discloses a resistant starch nanoparticle and a preparation method therefor, and the method includes: mixing a short-chain amylose mixed dispersion with konjac gum, gelatinizing to obtain a gelatinized short-chain amylose-konjac gum molecular solution, and then inducing the recrystallization of the gelatinized short-chain amylose-konjac gum molecular solution at −18-25° C. to obtain recrystallized starch particles, i.e., resistant starch nanoparticles. Chinese patent application No. CN115181772A discloses a method for preparing RS5 resistant starch by using waxy starch and α-linolenic acid as raw materials, and the method includes: dispersing elongated amylopectin with an alkaline solution, compounding with an α-linolenic acid solution to prepare a starch-lipid complex, and then preparing RS5 resistant starch. Both the two patents increase the resistant starch content of recrystallized products by introducing other functional components (konjac gum, α-linolenic acid, and the like) into a starch system, but the recrystallized products mainly have B-type and V-type crystalline structures, and the introduction of functional components significantly increases the processing cost and hinders the industrial production of resistant starch.

Moreover, conventional techniques for preparing crystalline starch typically require further auxiliary treatments such as annealing (CN117099942A, CN117643381A), toughening (CN115011652B), and magnetic induction (CN117327203A) to perfect a starch crystalline structure so as to increase the resistant starch content of a product, or other dietary fiber molecules are introduced to embed starch and protect the starch from enzymatic hydrolysis (CN117683248A). Such time-consuming and complex preparation processes greatly affect the production efficiency of resistant starch.

Furthermore, according to a method disclosed in a document (Liu Yangi, Yu Jiugao, Sun Xiuping. Preparation and Characterization of A-Type Starch Spherulites [J]. Journal of the Chinese Cereals and Oils Association, 2004. DOI: CNKI: SUN: ZLYX.0.2004-01-008.), an amorphous region of corn starch granules is hydrolyzed by mild acid hydrolysis of hydrochloric acid to obtain acid-hydrolyzed starch with relatively high crystallinity. The acid-hydrolyzed starch is dissolved and recrystallized by freezing to prepare B-type spherulites, and on the basis, further recrystallization is performed to obtain A-type starch spherulites. However, this method is not suitable for industrial production due to operation complexity and long crystallization time.

Therefore, there is an urgent need for developing a method for rapid and efficient preparation of A-type crystalline starch to solve technical problems and promote the industrial production of resistant starch.

### SUMMARY

### Technical Problems

Conventional methods for preparing crystalline starch have the deficiencies of cumbersome process, high cost, lack of environmental friendliness, and non-ideal crystallization effect, and the prepared crystalline starch is not A-type, but mostly B-type, V-type, or amorphous.

Methods for preparing A-type crystalline starch are not suitable for industrial production due to operation complexity and long crystallization time.

### Technical Solution

In order to solve the above problems, the present disclosure provides a method for efficiently crystallizing A-type starch. Specifically, the method of the present disclosure includes: gelatinizing and enzymatically debranching starch to obtain an enzymatic hydrolysate of starch; then pumping vapor of a solvent (ethanol, n-butanol, acetone, or the like) into the enzymatic hydrolysate of starch to induce starch crystallization, and centrifuging and drying to obtain A-type crystalline starch. The method of the present disclosure improves the efficiency of A-type starch crystallization, simplifies the preparation process of resistant starch, and shortens preparation time.

A first objective of the present disclosure is to provide a method for efficiently crystallizing A-type starch, and the method includes the following steps:
(1) Starch gelatinization:
dispersing starch in water and gelatinizing to obtain a gelatinized starch solution;
(2) enzymatic debranching:
adding pullulanase to the gelatinized starch solution and performing enzymatic hydrolysis to obtain an enzymatic hydrolysate of starch; and
(3) induced crystallization:
pumping vapor of a solvent into the enzymatic hydrolysate of starch at a rate of 80-120 mL/h to induce crystallization, after the crystallization, collecting a precipitate by centrifugation, and drying the precipitate to obtain A-type crystalline starch powder; where
the solvent is one or more of ethanol, n-butanol, and acetone.

In an embodiment of the present disclosure, the starch in the step (1) is waxy corn starch.

In an embodiment of the present disclosure, a ratio of starch to water in the step (1) is 5-15 g: 100 mL.

In an embodiment of the present disclosure, the gelatinization in the step (1) is stirring gelatinization in a boiling water bath, and specifically stirring gelatinization is performed at 90-100° C. and 100-500 rpm in a water bath for 5-40 min.

In an embodiment of the present disclosure, after the gelatinization in the step (1), a resulting mixture is cooled to 50-60° C.

In an embodiment of the present disclosure, a ratio of the starch in the step (1) to the pullulanase in the step (2) is 10 g: 1-2 mL.

In an embodiment of the present disclosure, an enzyme activity of the pullulanase in the step (2) is ≥1000 NPUN/g.

In an embodiment of the present disclosure, the enzymatic hydrolysis in the step (2) refers to an enzymatic hydrolysis reaction at 50-60° C. for 6-10 h, and further preferably at 58° C. for 8 h.

In an embodiment of the present disclosure, the pumping vapor of a solvent in the step (3) inactivates enzymes, so no additional enzyme inactivation step is required.

In an embodiment of the present disclosure, a ratio of the starch in the step (1) to the solvent in the step (3) is 10 g: 200-350 mL.

In an embodiment of the present disclosure, the vapor of a solvent in the step (3) is obtained by evaporating a liquid solvent.

In an embodiment of the present disclosure, a temperature for the induced crystallization in the step (3) is 40-80° C., and crystallization is completed when the solvent is completely added.

In an embodiment of the present disclosure, the centrifugation in the step (3) is performed at 2000-5000 rpm for 10-40 min.

In an embodiment of the present disclosure, the drying in the step (3) is performed at 40-50° C. for 4-20 h.

A second objective of the present disclosure is to provide A-type crystalline starch prepared by the method of the present disclosure.

In an embodiment of the present disclosure, the A-type crystalline starch has resistant starch content of more than 68% and a crystallinity of more than 75%.

A third objective of the present disclosure is an application of the A-type crystalline starch of the present disclosure in the fields of food processing and pharmaceutical preparation.

In an embodiment of the present disclosure, food processing includes the preparation of low-sugar food, weight-loss food, leisure food, food for special medical purposes, food additives, and the like.

In an embodiment of the present disclosure, the field of pharmaceutical preparation includes use of the A-type crystalline starch as a carrier for biochemical drugs.

A fourth objective of the present disclosure is to provide a method for improving anti-digestibility of resistant starch, where the A-type crystalline starch of the present disclosure is used.

### Beneficial Effects

(1) The method of the present disclosure simplifies the starch crystallization process and improves the preparation efficiency of resistant starch.
(2) The crystalline starch prepared by the present disclosure is A-type, and exhibits superior anti-digestibility.
(3) The A-type crystalline starch prepared by the present disclosure has the resistant starch content of more than 68% and the crystallinity of more than 75%.

### BRIEF DESCRIPTION OF THE DRAWINGS

FIG. 1 illustrates crystalline structures of starches prepared in Examples 1-3 and Comparative Examples 1-8.

### DETAILED DESCRIPTIONS OF THE EMBODIMENTS

Preferred embodiments of the present disclosure are described below, and it should be understood that the embodiments are intended to better explain the present disclosure and are not intended to limit the present disclosure.

### Test Methods

### 1. Characterizing a Crystal Form and Crystallinity of Recrystallized Starch by XRD

Collecting an XRD pattern of a sample to be tested at a 2θ scanning range of 4-40°, a scanning step size of 0.05° and a scanning speed of 2°/min.

### 2. Determining Resistant Starch Content of Recrystallized Starch by Englyst In Vitro Digestion Simulation

Dispersing 600 mg of crystalline starch in 20 mL of a sodium acetate buffer solution, after preheating to 37° C., adding 5 mL of a mixed enzyme solution containing pancreatin and amyloglucosidase (prepared by dispersing 18 g of pancreatin in 120 mL of water, fully mixing, and then mixing 90 mL of a supernatant with 4 mL of amyloglucosidase), starting digestion at 37° C., measuring the content of glucose G120 released in a digestion solution using a GOPOD kit after 120 min of digestion, and then calculating the resistant starch (RS) content of crystalline starch according to the following formula:

$$RS = \frac{M - 0.9 G 120}{M} \times 100 \%$$

where M is a total mass of starch.

### Raw Materials Used in the Examples

Waxy corn starch: purchased from Shandong Fuyang Biotechnology Co., Ltd.;
pullulanase: enzyme activity≥1000 NPUN/g, purchased from Sigma-Aldrich;
ethanol: 100%, purchased from Tianjin Zhiyuan Chemical Reagent Co., Ltd.;
n-butanol: 99.5%, purchased from Sinopharm Chemical Reagent Co., Ltd. (Shanghai); and
acetone: 99.5%, purchased from Sinopharm Chemical Reagent Co., Ltd. (Shanghai).

### Example 1

A method for preparing A-type crystalline starch, includes the following steps:
(1) Starch gelatinization:
10 g of waxy corn starch was dispersed in 100 mL of water and gelatinized in a boiling water bath (100° C.) with stirring at 300 rpm for 30 min, and then a resulting mixture was cooled to 58° C. to obtain a gelatinized starch solution;
(2) enzymatic debranching:
1 mL of pullulanase was added to the gelatinized starch solution and enzymatic hydrolysis was performed at 58° C. for 8 h to obtain an enzymatic hydrolysate of starch; and
(3) induced crystallization:
300 mL of ethanol was evaporated to obtain ethanol vapor; and
the ethanol vapor was pumped into the enzymatic hydrolysate of starch at a rate of 100 mL/h to induce crystallization at 58° C. for 3 h; and after the crystallization, a precipitate was collected by centrifugation at 4000 rpm for 10 min and dried at 45° C. for 12 h to obtain A-type crystalline starch powder.

### Example 2

The ethanol in the step (3) of Example 1 was replaced with n-butanol, and other conditions of Example 1 remained unchanged to obtain A-type crystalline starch powder.

### Example 3

The ethanol in the step (3) of Example 1 was replaced with acetone, and other conditions of Example 1 remained unchanged to obtain A-type crystalline starch powder.

### Comparative Example 1

A method for preparing crystalline starch, includes the following steps:
(1) Starch gelatinization:
10 g of waxy corn starch was dispersed in 100 mL of water and gelatinized in a boiling water bath (100° C.) with stirring at 300 rpm for 30 min, and then a resulting mixture was cooled to 58° C. to obtain a gelatinized starch solution;
(2) enzymatic debranching:
1 mL of pullulanase was added to the gelatinized starch solution, and enzymatic hydrolysis was performed at 58° C. for 8 h to obtain an enzymatic hydrolysate of starch; and the enzymatic hydrolysate of starch was placed in a boiling water bath for 10 min for enzyme inactivation, and then centrifuged at 4,500 rpm for 10 min to collect a supernatant; and
(3) crystallization:

The supernatant was recrystallized at 4° C. for 24 h; and after crystallization, a precipitate was collected by centrifugation at 4000 rpm for 10 min and dried at 45° C. for 12 h to obtain crystalline starch.

### Comparative Example 2

The ethanol in the step (3) of Example 1 was replaced with dimethyl sulfoxide, and other conditions of Example 1 remained unchanged to obtain crystalline starch powder.

### Comparative Example 3

A usage amount of ethanol in the step (3) of Example 1 was adjusted to 50 mL, and other conditions of Example 1 remained unchanged to obtain crystalline starch powder.

### Comparative Example 4

A method for adding the ethanol vapor in the step (3) of Example 1 was adjusted as follows:

300 mL of ethanol was directly added to the enzymatic hydrolysate of starch to induce crystallization, and other conditions of Example 1 remained unchanged to obtain crystalline starch powder.

### Comparative Example 5

The step (3) of Example 1 was adjusted as follows:
300 mL of ethanol was evaporated to obtain ethanol vapor; and
the enzymatic hydrolysate of starch was cooled to 25° C., and then ethanol vapor was pumped into the enzymatic hydrolysate of starch at a rate of 100 mL/h to induce crystallization; and after the crystallization, a precipitate was collected by centrifugation at 4000 rpm for 10 min and dried at 45° C. for 12 h to obtain crystalline starch powder.

### Comparative Example 6

The rate of ethanol vapor in the step (3) of Example 1 was adjusted to 200 mL/h, and other conditions of Example 1 remained unchanged to obtain crystalline starch powder.

### Comparative Example 7

The rate of ethanol vapor in the step (3) of Example 1 was adjusted to 50 mL/h, and other conditions of Example 1 remained unchanged to obtain crystalline starch powder.

### Comparative Example 8

The usage amount of ethanol in the step (3) of Example 1 was adjusted to 400 mL, and other conditions of Example 1 remained unchanged to obtain crystalline starch powder.

The obtained crystalline starch was subjected to a performance test, with test results as follows:

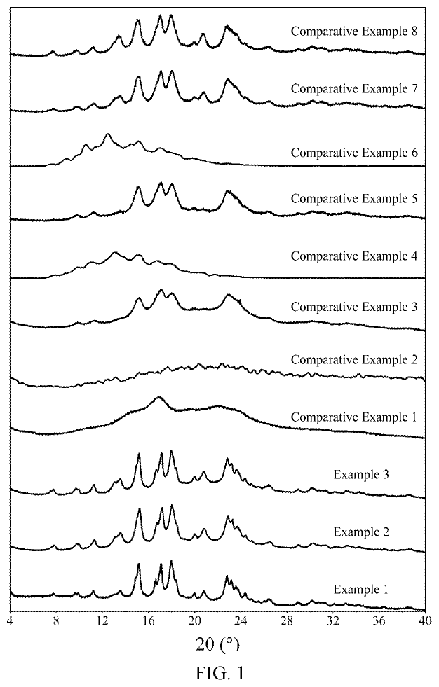

FIG. 1 illustrates crystalline structures of starches prepared in Examples 1-3 and Comparative Examples 1-8. It can be seen from FIG. 1 that:
(1) the crystalline starches prepared in Examples 1-3 and Comparative Examples 3-8 exhibited sharp diffraction peaks at 15°, 17°, 18°, and 23°, indicating that crystals thereof are A-type;
(2) the crystalline starch prepared in Comparative Example 1 has sharp diffraction peaks at 17°, 22°, and 24° respectively, indicating that crystals thereof are B-type;
(3) The crystalline starch prepared in Comparative Example 2 has an amorphous crystalline structure without obvious crystal diffraction peak observed.

Table 1 shows a yield, relative crystallinity, and resistant starch content of each crystalline starch. It can be seen from Table 1 that:
(1) The A-type crystalline starches prepared in Examples 1-3 have a relative crystallinity of more than 75%; yields of starch crystallization products are all more than 80%, and resistant starch content (RS %) of each crystalline starch is more than 68%; that is, the crystalline starches prepared in Examples 1-3 have high resistant starch content, and meet the application requirements of industrial resistant starch due to the simple preparation process, short preparation time, and high preparation efficiency;
(2) the crystalline starch prepared in Comparative Example 1 has a relative crystallinity of only 13.5%, indicating low crystallinity; a yield of a crystallization product is 73.6%, but the resistant starch content (RS %) is only 24.7%, thereby failing to meet application requirements of industrial resistant starch;
(3) the crystalline starch prepared in Comparative Example 2 has a relative crystallinity of only 5.3%, a yield of a crystallization product is 74.8%, and resistant starch content (RS %) of crystalline starch is only 1.9%, such that the crystalline starch cannot be used as resistant starch;
(4) the crystalline starches prepared in Comparative Examples 3-5 all have a low crystallinity, yield and resistant starch content, and cannot meet the application requirements of industrial resistant starch;
(5) excessively fast flow of vapor in Comparative Example 6 results in a low relative crystallinity (22.5%) and low resistant starch content (12.6%) of a product;
(6) a yield, crystallinity, and resistant starch content of crystalline starch in Comparative Example 7 are equivalent to those of Example 1, but the excessively slow flow of vapor significantly prolongs the crystallization time to 6 h, and hinders high-efficiency industrial production; and
(7) a yield, crystallinity, and resistant starch content of crystalline starch in Comparative Example 8 are equivalent to those of Example 1, but an increase in ethanol amount not only prolongs the crystallization time but also increases the raw material cost, thereby hindering high-efficiency industrial production.

TABLE 1
Yield, relative crystallinity, and resistant
starch content of each recrystallized starch

| | | | Relative | Resistant |
|---|---|---|---|---|
| | Yield | Crystal | Crystal- | Starch |
| Example | (%) | Form | linity (%) | Content (%) |
| Example 1 | 90.3 | A | 78.2 | 70.1 |
| Example 2 | 85.7 | A | 75.6 | 68.4 |
| Example 3 | 80.4 | A | 76.9 | 69.5 |
| Comparative Example 1 | 73.6 | B | 13.5 | 24.7 |
| Comparative Example 2 | 74.8 | Amorphous | 5.3 | 1.9 |
| Comparative Example 3 | 31.1 | A | 24.7 | 36.3 |
| Comparative Example 4 | 60.5 | A | 19.4 | 8.6 |
| Comparative Example 5 | 83.4 | A | 47.3 | 10.9 |
| Comparative Example 6 | 86.4 | A | 22.5 | 12.6 |
| Comparative Example 7 | 85.7 | A | 67.6 | 63.5 |
| Comparative Example 8 | 89.1 | A | 68.2 | 64.7 |

Although the present disclosure has been disclosed in preferred examples, but they are not intended to limit the present disclosure. Anyone skilled in the art can make various changes and modifications without departing from the spirit and scope of the present disclosure. Therefore, the scope of protection of the present disclosure should be defined by the claims.

What is claimed is:

1. A method for efficiently crystallizing A-type starch, comprising the following steps:
(1) starch gelatinization:
dispersing starch in water and gelatinizing to obtain a gelatinized starch solution;
(2) enzymatic debranching:
adding pullulanase to the gelatinized starch solution and performing enzymatic hydrolysis to obtain an enzymatic hydrolysate of starch; and
(3) induced crystallization:
pumping vapor of a solvent into the enzymatic hydrolysate of starch at a rate of 80-120 mL/h to induce crystallization; after the crystallization, collecting a precipitate by centrifugation, and drying the precipitate to obtain A-type crystalline starch powder; wherein
the solvent is one or more of ethanol, n-butanol, and acetone; and
a ratio of the starch in the step (1) to the solvent in the step (3) is 10 g: 200-350 mL.

2. The method for efficiently crystallizing A-type starch according to claim 1, wherein a ratio of the starch to water in the step (1) is 5-15 g: 100 mL.

3. The method for efficiently crystallizing A-type starch according to claim 1, wherein a ratio of the starch in the step (1) to the pullulanase in the step (2) is 10 g: 1-2 mL.

4. The method for efficiently crystallizing A-type starch according to claim 1, wherein the vapor of the solvent in the step (3) is obtained by evaporating a liquid solvent.

5. The method for efficiently crystallizing A-type starch according to claim 1, wherein a temperature for the induced crystallization in the step (3) is 40-80° C., and crystallization is completed when the solvent is completely added.

6. The method for efficiently crystallizing A-type starch according to claim 1, wherein the enzymatic hydrolysis in the step (2) refers to an enzymatic hydrolysis reaction at 50-60° C. for 6-10 hours.
